(19) United States
(12) Patent Application Publication
(10) Pub. No.: US 2023/0007769 A1
(43) Pub. Date: Jan. 5, 2023

(54) ELECTRONIC MODULE, METHOD OF MANUFACTURING ELECTRONIC MODULE, AND ENDOSCOPE

(71) Applicant: OLYMPUS CORPORATION, Tokyo (JP)

(72) Inventors: Junya YAMADA, Kawasaki-shi (JP); Hiroyuki MOTOHARA, Tokyo (JP)

(73) Assignee: OLYMPUS CORPORATION, Tokyo (JP)

(21) Appl. No.: 17/944,294

(22) Filed: Sep. 14, 2022

Related U.S. Application Data
(63) Continuation of application No. PCT/JP2020/011566, filed on Mar. 16, 2020.

(51) Int. Cl. H05K 1/02; H05K 1/18; H05K 3/28; H05K 3/10; A61B 1/05; A61B 1/018; A61B 1/00

(52) U.S. Cl. CPC H05K 1/0284; H05K 1/183; H05K 3/284; H05K 3/10; A61B 1/051; A61B 1/018; A61B 1/00098; H05K 2201/10121; H05K 2203/1305; A61B 1/0011

(57) ABSTRACT

An electronic module includes a three-dimensional wiring board including a cavity portion in which a bottom surface and four wall surfaces are formed, a plurality of electrodes being provided on the bottom surface, and a plurality of electronic components mounted on the plurality of electrodes and including a plurality of chip components and an image pickup module configured to pick up an image in an opening section direction of the cavity portion. A wall surface among the four wall surfaces that corresponds to a direction in which the plurality of chip components are arrayed is an inclined surface having an inclination with respect to the bottom surface.

### CROSS REFERENCE TO RELATED APPLICATION

This application is a continuation application of PCT/JP2020/011566 filed on Mar. 16, 2020, the entire contents of which are incorporated herein by this reference.

### BACKGROUND OF THE INVENTION

### 1. Field of the Invention

The present invention relates to an electronic module which is inexpensive and highly reliable although being downsized and highly integrated, a method of manufacturing an electronic module, and an endoscope.

### 2. Description of the Related Art

As portable terminals become popular, a trend toward downsizing of electronic components is accelerating nowadays, and a technology for providing predetermined functions for a substrate on which these electronic components are mounted to pursue downsizing is being actively proposed. Japanese Patent Application Laid-Open Publication No. 2016-86068, for example, discloses a technology for providing a main body part and a wiring pattern formed on an outer surface of the main body part, forming a recessed place in a mounting surface, and causing a substrate to also exert a reflector function of a light emitting device, thereby achieving downsizing. In addition, in Japanese Patent Application Laid-Open Publication No. 2016-86068, a three-dimensional circuit board is brought into contact with and mounted on a planar circuit board in a direction of a recessed portion to form a closed space, thereby ensuring a mounting space for components.

### SUMMARY OF THE INVENTION

An electronic module of an aspect of the present invention includes a three-dimensional wiring board including a cavity portion in which a bottom surface and four wall surfaces are formed, a plurality of electrodes being provided on the bottom surface, and a plurality of electronic components mounted on the plurality of electrodes and including a plurality of chip components and an image pickup module configured to pick up an image in an opening section direction of the cavity portion, in which a wall surface among the four wall surfaces that corresponds to a direction in which the plurality of chip components are arrayed is an inclined surface having an inclination with respect to the bottom surface.

A method of manufacturing an electronic module of an aspect of the present invention includes, in injection molding, subjecting a structure provided with a cavity portion to injection molding, the cavity portion being formed by four wall surfaces having an inclination in a runner direction and a bottom section spreading parallel to the runner direction, forming a wiring pattern provided from the bottom section in the cavity portion in a direction of a wall surface having the inclination, and mounting a plurality of electronic components on electrodes disposed on the wiring pattern.

An endoscope of an aspect of the present invention includes an electronic module including a three-dimensional wiring board including a cavity portion in which a bottom surface and four wall surfaces are formed, a plurality of electrodes being provided on the bottom surface, and a plurality of electronic components mounted on the plurality of electrodes and including a plurality of chip components and an image pickup module configured to pick up an image in an opening section direction of the cavity portion, a wall surface among the four wall surfaces that corresponds to a direction in which the plurality of chip components are arrayed being an inclined surface having an inclination with respect to the bottom surface, and an insertion section including the electronic module.

### BRIEF DESCRIPTION OF THE DRAWINGS

FIG. 1 is a magnified perspective view showing an electronic module according to a first embodiment of the present invention;

FIG. 2 is a side cross-sectional view showing the electronic module according to the first embodiment from a side;

FIG. 3 is a magnified perspective view of a principal part showing a configuration of a distal end portion of an insertion section in an endoscope to which the electronic module according to the first embodiment is applied;

FIG. 4 is a side cross-sectional view showing a cut-out part of the distal end portion of the insertion section in the endoscope to which the electronic module according to the first embodiment is applied;

FIG. 5 is a side view showing a manner in which solder paste is supplied into a cavity of the electronic module according to the first embodiment;

FIG. 6 is a side view showing a positional relationship between a cavity portion for an electronic module and a dispenser nozzle when it is supposed that the electronic module is formed with wall surfaces having no gradients;

FIG. 7 is a flowchart showing a method of manufacturing the electronic module according to the first embodiment;

FIG. 8 is an explanatory diagram showing steps of manufacturing the electronic module according to the first embodiment;

FIG. 9 is a diagram for explaining a relationship between a shape of the electronic module and laser light in a laser process for the electronic module;

FIG. 10 is a diagram for explaining a relationship between the shape of the electronic module and the laser light in the laser process for the electronic module;

FIG. 11 is a magnified perspective view of a principal part showing an inner configuration of a distal end portion of an insertion section in an endoscope to which an electronic module according to a second embodiment of the present invention is applied;

FIG. 12 is a side cross-sectional view showing a cut-out part of the distal end portion of the insertion section in the endoscope to which the electronic module according to the second embodiment is applied;

FIG. 13 is a perspective view showing an electronic module according to a third embodiment of the present invention;

FIG. 14 is a perspective view showing the electronic module according to the third embodiment from a rear surface side;

FIG. 15 is a side cross-sectional view showing the electronic module according to the third embodiment from a side;

FIG. 16 is a diagram for explaining a relationship between a shape of the electronic module and laser light in a laser process for the electronic module; and

FIG. 17 is a diagram showing an endoscope system to which the electronic modules of the first to third embodiments are applied.

### DETAILED DESCRIPTION OF THE PREFERRED EMBODIMENTS

Hereinafter, embodiments of the present invention will be explained with reference to the drawings.

Note that in each drawing to be used for the following explanation, respective constitutional elements may be varied in scale such that the respective constitutional elements have size enough to be recognizable in the drawings, and the present invention is not limited only to the number of the constitutional elements, the shapes of the constitutional elements, dimensional ratios of the constitutional elements, and a relative positional relationship among the respective constitutional elements depicted in the drawings.

<First Embodiment>

First, an example of placing and mounting a plurality of chip components and the like in a molded component including a cavity (recess) part in order to downsize an electronic module will be explained as a first embodiment of the present invention.

The electronic module can be used as an image pickup unit in a case in which an included electronic component is an image pickup module, for example. In this case, the electronic module can be utilized for various downsized cameras, and downsizing enables the electronic module to be incorporated into a wearable terminal, a distal end portion of an endoscope, or the like to pick up an image of a target.

Note that an outer portion of a box of a mold for a box-like molded product is a female die and called a cavity. Since the cavity has meanings of “a hollow, a hole, and a recess”, a recess in a molded product is herein called a cavity portion.

In the present embodiment, a molded component is formed by what is called the MID (molded interconnect devices) technology. Herein, the MID refers to a three-dimensional molded circuit component in which wirings for electric circuit are integrally formed in a surface of a three-dimensional molded product such as an injection molded product. The use of the MID technology enables wirings for circuit to be also formed in an inclined surface, a vertical surface, a curved surface, a through hole in a molded body, or the like different from a conventional two-dimensional circuit.

Note that the combined microfabrication technology disclosed particularly in Japanese Patent Application Laid-Open Publication No. 2008-159942 and Japanese Patent Application Laid-Open Publication No. 2011-134777 can be used for the MID. According to the combined microfabrication technology, what is called a 3D mounted device that enables fine patterning and bare chip mounting can be achieved by using the molded surface activation treatment technology, the laser patterning process, and the like for the MID technology for forming an electric circuit on a surface of an injection molded product.

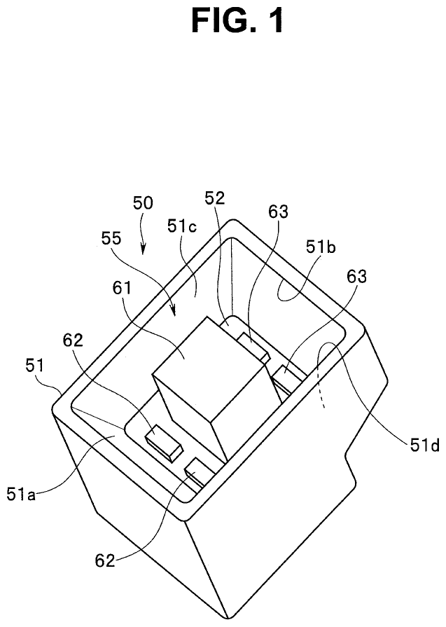

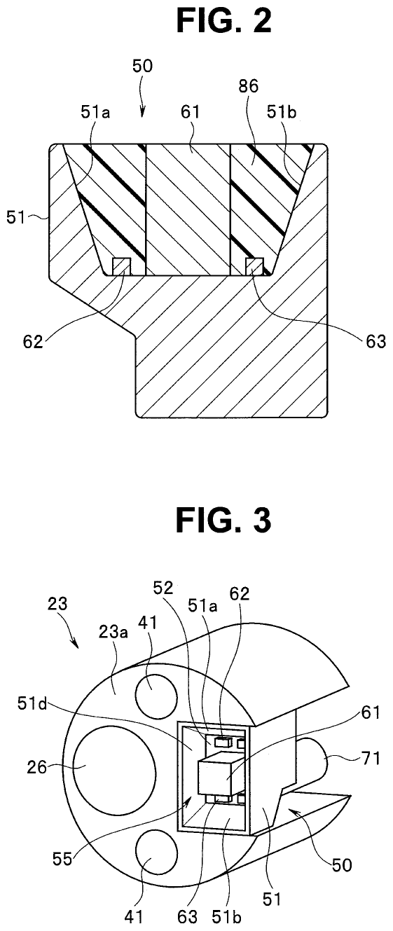

Hereinafter, an electronic module 50 according to the first embodiment of the present invention will be explained with reference to FIG. 1, FIG. 2, and the like. FIG. 1 is a magnified perspective view showing the electronic module according to the first embodiment of the present invention, and FIG. 2 is a side cross-sectional view showing the electronic module according to the first embodiment from a side.

As shown in FIG. 1 and FIG. 2, the electronic module 50 includes an MID frame part 51 including a cavity portion 55 formed by four wall surfaces 51a, 51b, 51c, and 51d that extend from a bottom section 52.

The electronic module 50 also has a wiring pattern and electrodes molded in a gradient direction of the wall surfaces 51a and 51b through a molded MID manufacturing process, for example, on the bottom section 52 of the cavity portion 55 of the MID frame part 51.

More specifically, the MID frame part 51 molded by an injection molding step, for example, is subjected to patterning and activation by radiating laser light to a surface of the molded product. Only a portion activated by performing plating is metallized to form a wiring pattern (not shown), and a plurality of electronic components can be mounted on lands (not shown) of the wiring pattern.

In the present embodiment, the electronic components to be mounted are chip components 62, 63, and the like such as a capacitor and a resistor, in addition to the image pickup module 61 described above.

Herein, the image pickup module 61 is a component having a relatively long length (that is, being relatively high) in a height direction standing from the bottom section 52 because a light receiving surface of an image pickup device is in parallel to a mounting surface, and an optical axis of an optical system staked on the image pickup device is in a direction substantially vertical to the mounting surface. Note that although the explanation herein is given using an example of the image pickup device, it goes without saying that a component having a relatively long length in the height direction other than the image pickup device can also be treated similarly. On the other hand, the chip components 62 and 63 are components having relatively short lengths (being relatively low) in the height direction.

In the present embodiment, the image pickup module 61 is mounted on a relatively central area of the bottom section 52 in order to ensure the degree of freedom for routing a wiring that deals with a control signal or an output signal in the cavity portion 55, or in order not to receive an optical influence from the cavity portion. The chip components 62 and 63 are disposed on the wiring pattern in the vicinity of the wall surface 51b at the bottom section 52 because of few wirings and absence of an influence from the cavity portion 55.

In a case of mounting chips on the relatively central area in the cavity in this manner, and filling the cavity with resin 86, and in a case in which a temperature property causes the resin to contract/shrink, it can be designed such that a balance between the wall surfaces and the chips considering the contraction/shrinkage is adjusted to reduce unbalanced stress to be imposed on the electronic components. In other words, a conceivable design is such that the cavity wall surfaces opposed to one another in the cavity portion of a three-dimensional substrate have a symmetric shape (having similar inclinations or gradients, for example) centering around a mounting area for electronic components such as sensors, thereby eliminating an imbalance of stress.

A distal end portion of an image pickup cable 71 for transmitting a signal for controlling the image pickup device in the image pickup module 61 or an image pickup signal generated in the image pickup device is also disposed in the electronic module 50.

The electronic module 50 according to the present embodiment can be mounted on various types of equipment because it is downsized and has a simple configuration. As shown in FIG. 3, for example, the electronic module in the present embodiment can also be mounted on a distal end portion 23 of an endoscope. The distal end portion 23 is a distal end portion of an insertion section of the endoscope not shown, and includes a rigid leading frame part 23a made of metal, for example. Note that the leading frame part 23a has an advantage of guarding a plurality of surfaces of the electronic module to make it less likely to receive an impact of a collision at the time of handling, for example. The material of the rigid leading frame part 23a is not limited to metal.

In the leading frame part 23a, an illumination optical system 41 that emits illumination light transmitted from a light source device by way of a light guide is disposed, and an opening section of a treatment instrument insertion channel 26 provided side by side with the light guide is disposed. Note that a predetermined treatment instrument can be inserted through the treatment instrument insertion channel 26. Downsizing of the electronic module enables a design with such a layout.

The image pickup cable 71 includes a cable main body (a covered portion) and an electric contact portion (a core portion) formed at a distal end portion of the cable main body although not shown, and is provided to extend through a flexible tube portion not shown, for example, of the insertion section of the endoscope not shown to transmit an image pickup signal generated by the image pickup device not shown in the image pickup module 61.

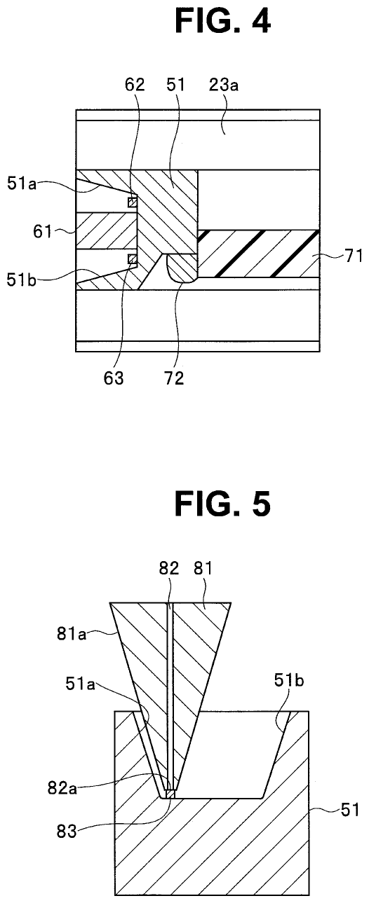

A signal line (a conductor electric wire, an electric conductor) of the image pickup cable 71 is soldered to a soldering portion 72 for an MID electrode as shown in FIG. 4. It can be said that a design that utilizes properties specific to the MID enables a conductor pattern to be formed in a manner extending around the three-dimensional structure in a direction of mounting the electronic module to a rear surface of the electronic module in this manner

Herein, FIG. 4 is a side cross-sectional view showing a cut-out part of the distal end portion of the insertion section of the endoscope in which the electronic module explained with reference to FIG. 3 has been incorporated.

The image pickup cable 71 includes the cable main body (the covered portion) and the electric contact portion (the core portion) formed at the distal end portion of the cable main body although not shown, and is provided to extend through a flexible tube portion not shown of the insertion section of the endoscope not shown. Further, the image pickup cable 71 is electrically connected by soldering to electrode portions (electric contact portions, lands for soldering) not shown in which a pattern has been formed from an electronic component mounting surface to a rear surface of the image pickup module 61 to enable communication of a control signal and an image pickup signal.

In this manner, when incorporating the distal end portion 23, a space for soldering can be provided without interfering with image pickup or increasing the thickness of the image pickup unit in the radial direction (a direction vertical to the optical axis or a direction vertical to a direction in which the endoscope is inserted). Further, the space for soldering can be ensured with ingenuity of the three-dimensional shape specific to the MID, which can improve workability and achieve downsizing.

A connector, for example, may be provided for such connection, which is effective in a case in which there is no space for arrangement, and can also be applied as a space for arranging the connector.

Further, a design in which the above-described dimension in the radial direction is reduced to increase easiness when inserting the endoscope can be obtained. A space for the light guide or the illumination optical system from the light source device provided in parallel is ensured, and a space for the treatment instrument insertion channel 26 is ensured, so that a light source which is bright and illuminates an appropriate range can be obtained. This contributes to obtaining a high-performance, sophisticated endoscope that is adaptable to complicated treatment. In other words, it is characterized by providing a space for electric connection by providing a recessed portion or a dent portion that is not influenced by the size of the opening of the cavity portion.

As described above, according to the electronic module 50 of the present embodiment, the entire circumference of the cavity portion 55 in the MID frame part 51 is covered by the four wall surfaces, for example. This is because the image pickup device and the chip components typically occupy a rectangular range on the mounting surface. In a case of filling the inside of the cavity portion 55 with sealing resin for stabilizing these components, the resin will not be flown to the outside.

Note that there may be three wall surfaces if the bottom surface is triangular, and some of sides of the walls of the cavity portion as in the present embodiment may be eliminated if sealing is performed taking a countermeasure against overflow of sealing resin. In addition, by forming a gradient for at least one surface among the above-described four wall surfaces such that the opening of the cavity portion is widened (in the present embodiment, the wall surfaces 51a and 51b have gradients), the opening is widened, molding of the MID frame part 51, generation of the wiring pattern, and mounting of the electronic components are facilitated, and improvement in reliability can also be expected. In a case of providing the electronic module of the present embodiment at the distal end portion of the endoscope, the wall surfaces are formed to have gradients with respect to the direction in which the insertion section of the endoscope is inserted.

The gradients of the wall surfaces 51a and 51b are set to be larger in inclination angle than the wall surfaces 51c and 51d. The gradients of the wall surfaces 51a and 51b are gradients for facilitating the shape of a mounting tool or laser processing which will be described later and facilitating pouring of resin, and are assumed to be more than or equal to approximately 5°, which is inclined more than a draft of the wall surfaces 51c and 51d in typical injection molding of less than or equal to 3°.

Large inclination angles of the wall surfaces 51a and 51b facilitate laser processing or resin filling. However, the electronic module will be increased in size as a whole. Thus, by providing a gradient for only a wall surface in a necessary direction, the influence can be reduced. In a case in which the mounting surface or the light receiving surface of the image pickup device is rectangular, for example, the influence of optical vignetting is reduced, and size increase of the electronic module can be minimized by performing mounting with the longitudinal direction of the device light receiving surface or the mounting surface conformed to a wall surface having a larger inclination angle.

By inclining some wall surfaces among the plurality of wall surfaces constituting the cavity portion, it can be designed such that stress produced when sealing resin contracts/shrinks depending on the temperature escapes to the opening section. In the present embodiment, the wall surfaces 51a and 51b are provided with a stress distribution function achieved by the inclination. In this manner, an electronic module having excellent processability and reliability can be obtained by virtue of the gradients provided for the wall surfaces 51a and 51b.

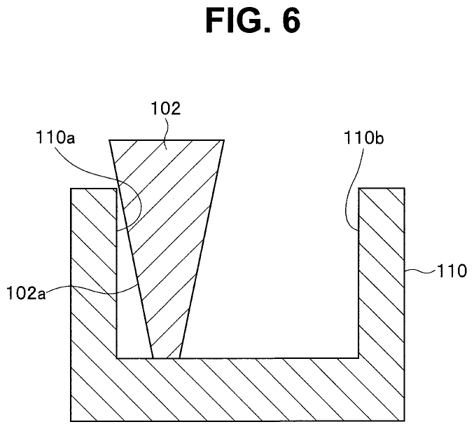

Herein, a method of mounting each component on the electronic module with the cavity portion through use of the MID in the present embodiment is explained, but solder paste first needs to be applied to correct positions for soldering mounted components. Thus, supply of solder paste for soldering the chip components 62 and 63 disposed in the vicinity of the wall surfaces 51a and 51b will be explained with reference to FIG. 5 and FIG. 6.

FIG. 5 is a side cross-sectional view showing a dispenser nozzle for supplying solder paste into the cavity of the electronic module according to the first embodiment, and also showing a manner in which the solder paste is supplied.

As shown in FIG. 5, a dispenser nozzle 81 for soldering corresponding to the electronic module 50 of the present embodiment is a precision nozzle having a nozzle inner-diameter section 82, and a taper 81a having a predetermined angle is formed at a distal end portion. Note that the gradation angle of the above-described wall surface 51a in the MID frame part 51 of the electronic module 50 is set at an angle corresponding to the angle of the above-described taper 81a.

When supplying the solder paste 83 to an electrode on the wiring pattern in the cavity portion 55, a distal end 82a of the nozzle inner-diameter section 82 in the dispenser nozzle 81 is positioned on a predetermined electrode, and then the solder paste 83 is applied from the distal end 82a, as shown in FIG. 5.

As described above, the distal end portion of the dispenser nozzle 81 has the taper 81a, that is, presents a shape spreading out toward a proximal end portion. The angle of the taper 81a corresponds to the gradation angle of the wall surface 51a. Thus, in the case of supplying the solder paste to an electrode corresponding to the chip component 62 disposed in the vicinity of the wall surface 51a on the bottom section 52 of the cavity portion 55, solder can be smoothly supplied to a place close to edges of the walls of the mounting surface without the distal end portion of the dispenser nozzle 81 being interfered with by the wall surface 51a.

On the other hand, if it is supposed that the electronic module is formed with wall surfaces having no gradients as shown in FIG. 6, a distal end surface of a dispenser nozzle 102 and a wall will interfere with each other to create a useless space in the vicinity of the wall at the bottom section in the cavity portion where solder cannot be applied. Even in the case in which an electronic module is formed with wall surfaces having no gradients, a predetermined thickness should be ensured considering the strength of the wall surfaces. Thus, when a mounting area for electronic components is ensured, the entire size is increased. On the other hand, in the case in which the walls of the MID have gradients, the MID is molded with the thickness of the bottom section being ensured although the thickness at the top is thinner than the thickness proximate to the bottom section, which is also advantageous in terms of strength.

<Steps of Manufacturing Electronic Module 50>

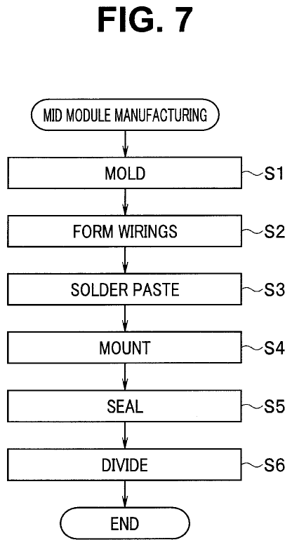

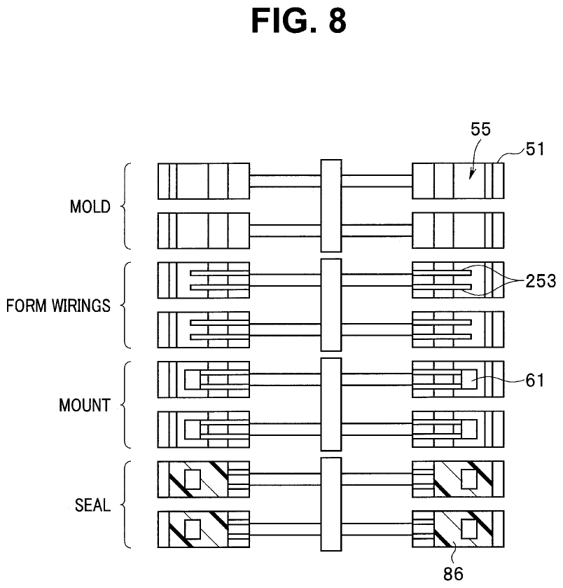

Next, steps of manufacturing the electronic module 50 will be explained with reference to FIG. 7 and FIG. 8.

FIG. 7 is a flowchart showing a method of manufacturing an electronic module according to the first embodiment, and FIG. 8 is an explanatory diagram showing steps of manufacturing the electronic module. Note that FIG. 1 and the like shall be referred to for reference numerals of wall surfaces shown here, and only main regions are denoted by reference numerals to prevent the drawings from becoming complicated.

In the case of manufacturing the electronic module 50, first, a predetermined resin material is set in a mold, and in a case of performing multi-cavity molding in injection molding, injection molding is performed on the MID frame part 51 provided with the opening section of the cavity portion 55 formed by a plurality of wall surfaces (51a, 51b, 51c, and 51d) including the wall surfaces 51a and 51b having gradients as well as the bottom section 52 in a direction perpendicular to a runner direction and a direction perpendicular to a direction in which multi-cavity molds are arrayed (step S1).

Next, a wiring pattern is formed on a surface of the bottom section 52 of the cavity portion 55 where the gradients of the wall surfaces 51a and 51b are formed (step S2).

In step S2, the MID frame part 51 molded in the above-described injection molding step, for example, is irradiated with laser light at a surface of a molded product to perform patterning and activation. Only an activated portion by performing plating is metallized to mold a wiring pattern 253 and to form a plurality of electrodes on the wiring pattern.

Next, solder paste for mounting a corresponding one of the electronic components (the image pickup module 61 and the chip components 62, 63) on each of the plurality of electrodes molded on the wiring pattern is supplied (step S3).

When the solder paste is supplied to the electrodes on the wiring pattern in the cavity portion 55 in step S3, the distal end 82a of the nozzle inner-diameter section 82 in the dispenser nozzle 81 is positioned on a predetermined electrode, and then the solder paste 83 is applied from the distal end 82a, as shown in FIG. 5.

Next, the electronic components such as the image pickup module 61 and the chip components 62, 63 are mounted on corresponding electrodes (step S4).

Next, in the cavity portion 55, a space formed by the above-described wall surfaces 51a, 51b, 51c, and 51d and the above-described plurality of mounted components (the image pickup module 61 and the chip components 62, 63) is filled with the predetermined resin 86 to perform sealing (step S5; see FIG. 2).

When sealing with resin is completed in step S5, a separating step (division) is executed (step S6), and the electronic module 50 on which the above-described respective electronic components have been mounted is completed. Note that the separating step may not be performed at the end of the process, but may be performed immediately after the molding step, for example. The separating step is carried out at appropriate timing by overviewing the entire electronic module manufacturing process.

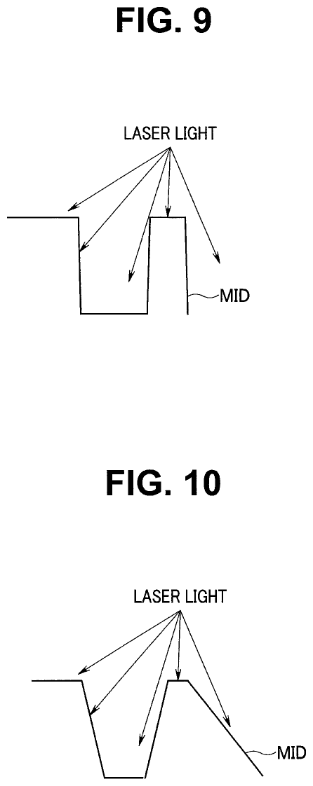

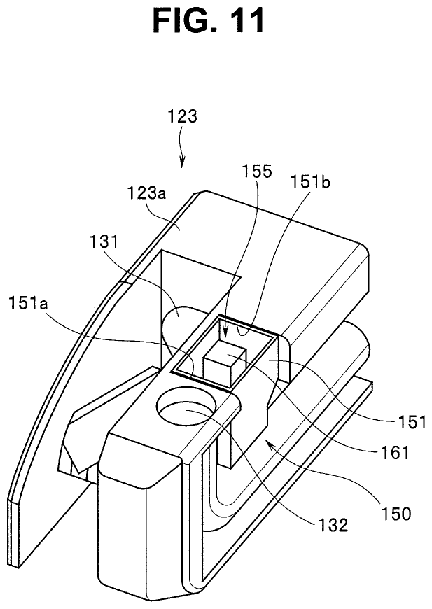

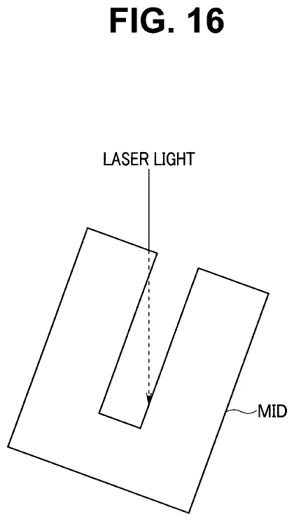

Next, an effect exerted by providing the predetermined gradients for the wall surfaces 51a and 51b of the MID frame part 51 as described above will be explained with reference to FIG. 9, FIG. 10, and FIG. 16.

FIG. 9, FIG. 10, and FIG. 16 are diagrams for explaining a relationship between a shape of the electronic module and laser light radiation for forming an electric connection pattern in a laser process for the electronic module.

In a case of manufacturing the electronic module 50 as in the present embodiment through the laser process, it is ideally desirable that laser should be radiated while maintaining an appropriate angle with respect to a resin surface on which an electric conductor pattern (wiring pattern) is to be formed. However, with sheer wall surfaces as in FIG. 9, the wall portions cast shadow to unable laser radiation, so that a pattern that runs on the wall surfaces from the bottom section of the cavity cannot be formed.

A configuration in which the wall surfaces are thus graded as in the present embodiment enables laser light to scan in directions of arrows as shown in FIG. 10, and enables a wiring pattern continuous from the component mounting area at the bottom section of the cavity to be routed to the outside of the cavity portion with little effort by one scan. In this manner, the laser process can be simplified by the gradients of the wall portions to manufacture a highly reliable, inexpensive module with reliable wirings.

It is ideal that the target resin surface should be irradiated with laser at a radiation angle of 90°, and deteriorates in quality as the radiation angle becomes smaller.

In a case in which the cavity portion of the electronic module is formed with wall surfaces having no gradients and the cavity is deep as shown in FIG. 16, for example, the wall surfaces cause vignetting of laser light, that is, the degree of freedom in manufacturing is low.

In contrast, the electronic module 50 as in the present embodiment, for example, the wall surfaces 51a and 51b among the four wall surfaces that form the cavity portion 55 are provided with gradients as described above. Thus, the presence of the gradients increases the degree of freedom in shape of the cavity portion 55. In addition to laser scanning, the MID member side may be moved to change a radiation position to create a wiring pattern, or these may be combined. In order to radiate laser even in a direction opposite to the mounting area, a plurality of laser light sources may be used, or an inclination of the components may be changed.

<Second Embodiment>

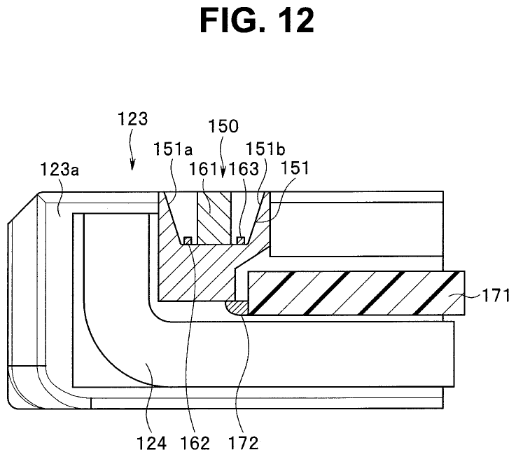

Next, a second embodiment of the present invention will be explained. FIG. 11 is a magnified perspective view of a principal part showing an inner configuration of a distal end portion of an insertion section in an endoscope according to the second embodiment of the present invention, and FIG. 12 is a side cross-sectional view showing a cut-out part of the distal end portion of the insertion section.

As shown in FIG. 11, in the present second embodiment, an electronic module 150 surrounded by an MID frame part 151 as shown in FIG. 1 is arranged so as to pick up an image of a side surface when the endoscope is inserted.

An illumination optical system 132 that radiates illumination light transmitted from a light source device by way of a light guide 124 and an image pickup module 161 are disposed at a distal end portion 123 of the insertion section not shown.

The electronic module 150 at the distal end portion 123 is placed in a recess in a rigid leading frame part 123a (made of metal, for example), and is advantageously less likely to receive an impact of a collision at the time of handling, for example, with a plurality of surfaces of the electronic module being guarded.

Further, a treatment instrument insertion channel 131 is provided in the rigid leading frame part 123a side by side with the electronic module 150, so that a predetermined treatment instrument can be inserted. Since the electronic module 150 (and the illumination optical system 132) is disposed in this manner at a position at which how a treatment instrument moves in a direction different from the direction in which the endoscope is inserted can be checked, the electronic module 150 needs to be downsized together with the treatment instrument insertion channel 131.

It is important to reduce a dimension particularly in a direction perpendicular to the direction in which the endoscope is inserted in order to reduce pain in a case of inserting the insertion section into a body cavity of a subject and to also enable insertion through a small hole in another inspection. Thus, a layout is such that wall surfaces 151a and 151b having gradients conform to the direction in which the endoscope is inserted.

What is called a raising base (forceps elevator) for treatment instrument is disposed in front of the treatment instrument insertion channel 131, so that a treatment instrument inserted through the treatment instrument insertion channel 131 can change an orientation of a distal end portion of the treatment instrument in an operation of the raising base. A downsized endoscope can also be protruded further from the distal end portion. Such an insertion channel is a member made of a rigid material such as metal or resin so as to be prevented from being deformed when the treatment instrument having excellent operability enters/exits or the orientation of the distal end portion of the treatment instrument is changed on the raising base.

When changing the orientation, an elastic member needs to be pulled using a wire, for example, and it is important that deformation should be prevented even when a pulling force is received, thereby controlling the treatment instrument to be located at a correct position. The electronic module is arranged side by side with the insertion channel 131 in the direction perpendicular to the insertion direction (which is also a pulling direction) so as not to be influenced by the force at this time or a mechanism arrangement.

FIG. 12 shows that a space for providing a soldering portion 172 that solders a wiring from a cable wire that controls an image pickup device and the like and communicates an image pickup signal is left in the electronic module 150 similarly to FIG. 4. In this manner, an arrangement in which the cable wire having effects such as shielding is brought as close as possible to the electronic components can achieve a highly reliable, high-definition design that is less likely to be influenced by noise or the like.

Herein, a dent part (recessed portion) is provided in a portion opposite to the mounting surface of the electronic module such that a cable 171 can be brought as close as possible to the electronic module 150. Downsizing is achieved with ingenuity of the three-dimensional shape specific to the MID.

Although the example of placing a bulge of solder in the dent part has been shown in the first embodiment, the dent part herein is used as a space for placing the cable itself. A soldering portion for the cable and electrodes of the electronic module 150 will be explained in a third embodiment.

Cable wiring can be performed without interfering with the layout of the above-mentioned pulling mechanism. In this manner, by providing a downsized electronic module which is a feature of the present invention, a side-viewing endoscope can be downsized. Further, reliable treatment instrument control and image pickup device control enables a highly reliable, easy-to-use endoscope product to be provided.

<Third Embodiment>

Next, the third embodiment of the present invention will be explained. FIG. 13 to FIG. 15 show the third embodiment of the present invention. Using these drawings, a wiring pattern is also illustrated herein in an easy-to-understand manner so as to also explain the first embodiment and the second embodiment described above together. In other words, portions not depicted in the drawings according to the first embodiment and the second embodiment described above shall be equivalent to content which will be explained herein.

As shown in FIG. 13, the third embodiment includes an incorporating part 256 for facilitating incorporation of an electronic module 250 into a distal end portion of an endoscope or the like.

The incorporating part 256 includes a recessed portion so as to enable positioning through use of a screw, for example. The incorporating part 256 is provided at a portion molded in an extended part provided in a direction identical to a gradient of a cavity portion in the electronic module 250, and conforms to the direction in which the endoscope is inserted, for example, thereby reducing a radial dimension which is an obstacle at the time of insertion.

Since the incorporating part 256 can be handled in a manner not to touch a metal wiring pattern in an incorporating operation to produce a defect such as a crack, a design is obtained in which handling when a product is manufactured or when the module is inspected is improved.

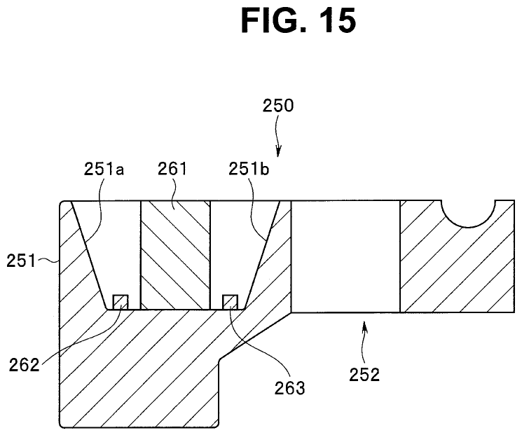

FIG. 15 is a side cross-sectional view of the electronic module according to the present third embodiment, and the relationship between the gradients of the cavity portion of a frame member (MID) and electronic components is also provided making effective use of the mounting surface in a direction in which the gradients are present similarly to the first and second embodiments.

An image pickup module 261 which is a stacked lens or the like having a height with respect to the mounting surface is sealed by filling the cavity with sealing resin according to necessity. This enables manufacturing which is also preferable in terms of improvement of reliability in which management is performed such that when filling the cavity with resin, an occurrence of air bubbles, for example, can be prevented by flowing resin along the gradient part, overflowing or spillover is prevented, and the amount of sealing resin that fills the periphery of the image pickup module 261 becomes substantially uniform.

The present embodiment can also have a watertight structure by means of resin, a material of an optical system, or ingenuity of design. The electronic module can be developed to various applications because of downsizing.

The direction of the extended part extended in the direction in which the walls having gradients are arrayed is also a direction in which the runner explained with reference to FIG. 8 extends. Further, the extended part presents a shape extended in the insertion direction when incorporating the electronic module (image pickup unit) into the distal end portion of the endoscope or the like, and contributes to downsizing for entering a narrow place. In other words, by providing the incorporating part, a design is obtained in which the length in the direction perpendicular to the insertion direction will not be long.

At the time of injection molding, resin is injected in the direction of the runner. It is generally known that a coefficient of linear expansion in a flow direction of resin containing a filler is small with respect to a right angle direction. The walls of the cavity are at right angles to the flow direction, that is, the bottom surface of the cavity in which the electronic components are mounted is parallel to the flow direction of resin. Thus, the coefficient of linear expansion is small, and is advantageous in terms of reliability.

In a case of mounting the electronic components on a relatively central area in the cavity, and filling the cavity with resin, and assuming a case in which a temperature property causes contraction/shrinkage of resin, the respective opposite wall surfaces of the cavity may have symmetric shapes centering around the mounting area for the electronic components such as sensors in the cavity portion of a three-dimensional substrate. It can therefore be expected that a balance between forces to be exerted by the contraction/shrinkage on the wall surfaces and the electronic components is adjusted to reduce unbalanced stress on the electronic components.

When such an extended part is present, a wiring pattern from the cavity portion to the cable (see FIG. 4) along the wall surfaces having gradients is elongated to degrade the quality of signals. Thus, a through hole 252 is provided such that wirings on the rear side of the mounting surface for the image pickup device, for example, can be routed by a short distance. A pattern 253 to the soldering portion can be produced with a short wiring using the through hole 252, and a scan range with laser light continuously radiated along the pattern is simplified. Such ingenuity facilitates manufacturing.

In other words, a pattern including a through hole that extends from the front surface to the rear surface of the three-dimensional substrate is provided in a region other than the cavity portion of the three-dimensional substrate, and intended for connecting terminals of a sensor mounting area and external terminals through a surface of the above-described opening section is formed. The through hole saves the effort of wiring step production to facilitate manufacturing, and shortens wirings themselves to reduce an influence caused by noise or the like entering the signal line when the module is inspected or actually used. Some of the wirings are less likely to run into each other at the portion where the through hole is formed, resulting in favorable handling and contributing to improved productivity.

Further, as shown in FIG. 15, it is also understood that a frame member includes a flat bottom section, and the electronic module 250 has a structure to be easily laid on a working table or the like when the electronic module 250 is handled with the extended part gripped.

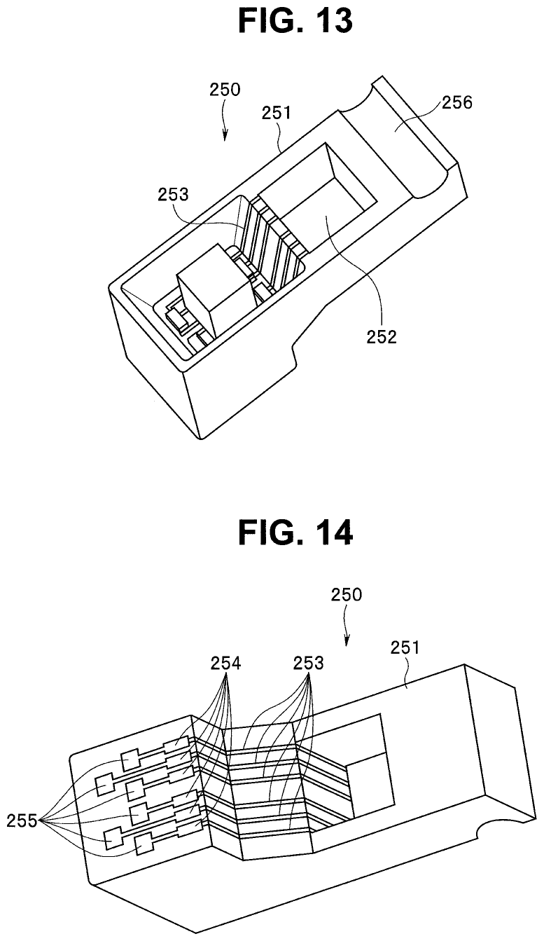

As is also clear from the perspective view of a rear surface of the electronic module according to the present third embodiment shown in FIG. 14, the third embodiment enables how the wiring pattern 253, explanation of which is omitted in the first and second embodiments, is routed from the cavity portion to be checked.

FIG. 14 also illustrates in conjunction with FIG. 13 how wirings extending upward from the mounting surface along the gradient part continue to soldering lands 254. It is assumed that the first and second embodiments have similar wirings.

In particular, it is assumed that the cable in the second embodiment (FIG. 11) is soldered to the cable connection electrodes (soldering lands) 254 illustrated in FIG. 14.

Soldering lands in the first embodiment should be provided at a portion of a surface substantially perpendicular to the bottom surface of the module following the wirings toward the electronic circuit with reference to FIG. 14.

Inspection electrodes 255 are provided on the bottom surface of the module equivalent to the rear surface of the mounting surface such that the module can be placed on an inspection table or the like to verify functions and performance of the image pickup device and the like. This enables an inspection including an image pickup signal to be performed without shielding or the like of an image of a target caused to enter the image pickup device or the like during the inspection.

In other words, the pattern extended to the rear side of the image pickup device in a viewing field direction for signals of the image pickup device and the like is electrically connected to inspection terminals provided on a parallel flat surface on the rear side of the above-described sensor mounting area, thereby making it less likely to be influenced by an inspection jig, a circuit, wirings, and the like in an inspection step, and enabling a check pin and the like to be applied reliably.

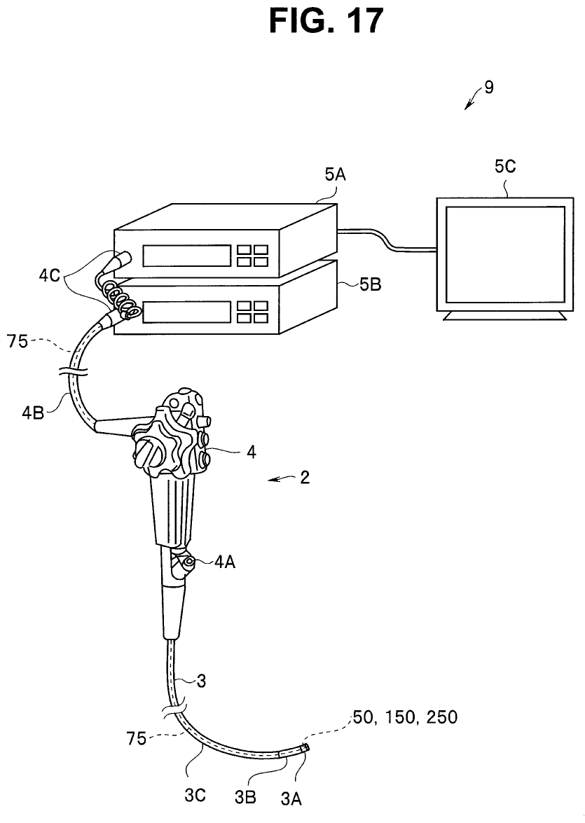

Next, an endoscope system to which the electronic modules of the first to third embodiments are applied will be explained with reference to FIG. 17.

As shown in FIG. 17, an endoscope system 9 includes the endoscope 2, a processor 5A, a light source device 5B, and a monitor 5C. The endoscope 2 inserts the insertion section 3 into a body cavity of a subject to pick up an image of the inside of the body of the subject, and outputs an image pickup signal. In other words, the endoscope 2 includes any of the electronic modules (image pickup units) 50, 150, and 250 at a distal end portion of the insertion section 3.

The operation section 4 provided with various buttons for operating the endoscope 2 is disposed on a proximal end side of the insertion section 3 of the endoscope 2. The operation section 4 includes a treatment instrument insertion port 4A of a channel through which a treatment instrument such as a biological forceps, an electric cautery, and an inspection probe is to be inserted into the body cavity of the subject. A channel opening section is provided at the distal end.

The insertion section 3 is composed of a distal end portion 3A at which the image pickup apparatus 1 is disposed, a bendable bending portion 3B provided in a manner coupled to the proximal end side of the distal end portion 3A, and a flexible tube portion 3C provided in a manner coupled to the proximal end side of the bending portion 3B. The bending portion 3B is bent by an operation of the operation section 4.

A signal cable 75 connected to the image pickup apparatus 1 at the distal end portion 3A is inserted through a universal cord 4B disposed on the proximal end portion side of the operation section 4.

The universal cord 4B is connected to the processor 5A and the light source device 5B via connectors 4C. The processor 5A controls the endoscope system 9 as a whole, and performs signal processing on an image pickup signal outputted from the image pickup apparatus 1 to output an image signal. The monitor 5C displays the image signal outputted from the processor 5.

The light source device 5B includes a white LED, for example. White light emitted from the light source device 5B is guided to an illumination optical system (not shown) of the distal end portion 3A via a light guide (not shown) inserted through the universal cord 4B to illuminate the subject.

Since the endoscope 2 includes the downsized image pickup apparatus 50, 150, or 250 at the distal end portion of the insertion section, the endoscope 2 can be reduced in diameter. As described above, by configuring the endoscope such that the image pickup unit (electronic module) and the channel are arranged at the distal end portion so as to be perpendicular to the direction in which the endoscope is inserted, the image pickup unit is less likely to receive stress caused by a member taken in/out of the channel part, and the three-dimensional wiring board including the cavity portion in which the bottom surface of the image pickup unit and the plurality of walls are formed, and the plurality of electronic components mounted on electrodes provided at the bottom surface, and the like are safely protected. Since a wall corresponding to the direction in which the above-described plurality of electronic components are arrayed among the plurality of walls of the above-described cavity portion is inclined with respect to the bottom surface of the cavity portion, and is in a direction substantially perpendicular to the direction in which the adjacent channel is arrayed, the distal end of the endoscope can be narrowed to facilitate insertion.

The present invention is not limited to the embodiments described above, and various changes, modifications, and the like can be made within a range not changing the gist of the present invention. For example, application of replacing the portion explained as the endoscope with another camera such as a consumer camera, an industrial camera, an on-vehicle camera, or a surveillance camera can be performed. In other words, by exploiting the characteristics of downsizing of the present invention, a space including a cable wiring that controls the image pickup unit and receives a signal from the image pickup unit can be saved in a direction perpendicular to a direction in which the wirings are pulled out. Thus, even in a case of a system or a layout in which a control circuit that controls an image pickup unit arranged in a narrow space is arranged away from the image pickup unit, a sophisticated image pickup apparatus can be incorporated. Consequently, since many image pickup units are mounted on an automobile for which there is a need to pick up images of various places without blind spots outside or inside the vehicle, downsizing including even wirings as in the present invention is important, and facilitates design at the time of incorporation. The present invention can also be applied to a portable terminal required to be downsized and reduced in weight for portability, a network terminal such as an AI speaker required to be placed at a small spot, IoT consumer electronics, and a watching camera that watches everyday life of a target to assure the security of the target. Further, the image pickup unit is easily incorporated into a movable body, such as a robot (including a vacuum cleaner and the like) or a drone, in which downsizing, weight reduction, and further, the center of gravity of the apparatus, and balance are also important since a moving function is important.

The electronic module and the three-dimensional wiring board including the cavity portion for the image pickup unit in the above description are not necessarily limited to those produced by the MID technology through injection molding, but may be produced by processing with a 3D printer or cutting machining, for example. The material is not limited to resin, but ceramic or glass epoxy may be used.

What is claimed is:

1. An electronic module comprising:
a three-dimensional wiring board including a cavity portion in which a bottom surface and four wall surfaces are formed, a plurality of electrodes being provided on the bottom surface; and
a plurality of electronic components mounted on the plurality of electrodes and including a plurality of chip components and an image pickup module configured to pick up an image in an opening section direction of the cavity portion, wherein
a wall surface among the four wall surfaces that corresponds to a direction in which the plurality of chip components are arrayed is an inclined surface having an inclination with respect to the bottom surface.

2. The electronic module according to claim 1, wherein the image pickup module is mounted on a central area of the bottom surface, and the plurality of chip components are mounted around the image pickup module.

3. The electronic module according to claim 2, wherein a wiring pattern mounted on the bottom surface along the wall surface which is inclined extends to a rear surface of a mounting surface in the electronic components.

4. The electronic module according to claim 1, further comprising resin that fills a space formed by the cavity portion and at least one electronic component among the plurality of electronic components.

5. The electronic module according to claim 1, wherein at least part of the electronic module is arranged in a metal enclosure.

6. The electronic module according to claim 5, further comprising an incorporating part disposed in a direction of the inclination of the wall surface which is inclined, and configured to fix the electronic module when the electronic module is arranged in the metal enclosure.

7. The electronic module according to claim 1, wherein
the electronic module is disposed at a distal end portion of an endoscope,
the distal end portion of the endoscope includes a channel, and
the electronic module and the channel are arranged to be perpendicular to a direction in which the endoscope is inserted, and a gradient direction of the cavity portion of the electronic module is a direction substantially perpendicular to a direction in which the channel which is adjacent to the electronic module is arrayed.

8. The electronic module according to claim 7, wherein
the channel includes a movable part, and
the movable part is a forceps raising base.

9. A method of manufacturing an electronic module, comprising:
in injection molding, subjecting a structure provided with a cavity portion to injection molding, the cavity portion being formed by four wall surfaces having an inclination in a runner direction and a bottom section spreading parallel to the runner direction;
forming a wiring pattern provided from the bottom section in the cavity portion in a direction of a wall surface having the inclination; and
mounting a plurality of electronic components on electrodes disposed on the wiring pattern.

10. The method of manufacturing an electronic module according to claim 9, further comprising filling a space with resin, the space being formed by the cavity portion and at least one electronic component among the plurality of electronic components.

11. An endoscope comprising:
an electronic module including a three-dimensional wiring board including a cavity portion in which a bottom surface and four wall surfaces are formed, a plurality of electrodes being provided on the bottom surface, and a plurality of electronic components mounted on the plurality of electrodes and including a plurality of chip components and an image pickup module configured to pick up an image in an opening section direction of the cavity portion, a wall surface among the four wall surfaces that corresponds to a direction in which the plurality of chip components are arrayed being an inclined surface having an inclination with respect to the bottom surface; and
an insertion section including the electronic module.
